(19) United States
(12) Patent Application Publication
(10) Pub. No.: US 2017/0072085 A1
(43) Pub. Date: Mar. 16, 2017

(54) MICROFLUIDIC DELIVERY SYSTEM AND CARTRIDGE

(71) Applicant: The Procter & Gamble Company, Cincinnati, OH (US)

(72) Inventors: Dana Paul GRUENBACHER, Fairfield, OH (US); Simon DODD, West Linn, OR (US); David S. HUNT, San Diego, CA (US); Joseph Edward SCHEFFELIN, San Diego, CA (US); Faiz Feisal SHERMAN, Mason, OH (US)

(21) Appl. No.: 14/855,677

(22) Filed: Sep. 16, 2015

(51) Int. Cl. A61L 9/12; A61L 9/14

(52) U.S. Cl. CPC A61L 9/127; A61L 9/145; A61L 9/12; A61L 2209/132; A61L 2209/133

(57) ABSTRACT

A cartridge for a microfluidic delivery system is provided. The cartridge includes a reservoir for containing a fluid composition. The cartridge includes a capillary tube having a first end portion and a second end portion opposing the first end portion. The first end portion of the capillary tube is in fluid communication with the reservoir. The second end portion of the capillary tube is operatively connected with the reservoir. The cartridge includes a restriction member that is in fluid communication with the capillary tube and that is configured to restrict fluid flow. The cartridge comprises a microfluidic delivery member connected with and in fluid communication with the reservoir, wherein the microfluidic delivery member comprises a die having at least one nozzle.

### FIELD

The present disclosure generally relates to systems for delivering a fluid composition into the air or onto a surface, and, more particularly, relates to microfluidic delivery systems and cartridges for delivering fluid compositions into the air.

### BACKGROUND

Various systems exist to deliver fluid compositions, such as perfume compositions, into the air by energized (i.e. electrically/battery powered) atomization. Recent attempts have been made to deliver fluid compositions, such as perfume compositions, into the air using microfluidic delivery technology, including thermal and piezo inkjet technology. Thermal and piezo inkjet cartridges may include a die having a nozzle for dispensing fluid.

Inkjet cartridges are often designed to release fluid downward, in the same direction as the gravitational force acting on the ink. However, in the case of releasing liquid compositions, such as perfume compositions, into the air, delivering the liquid composition up, against the force of gravity, may be beneficial. One issue with configuring a microfluidic delivery member to release a liquid composition into the air is that air bubbles can clog the nozzle and prevent liquid composition from being released through the nozzle.

One method of feeding a fluid composition up to a die includes the use of capillary tube, using capillary action to deliver the fluid composition to the die. However, if the cartridge is dropped or struck, air from the environment adjacent the nozzles may enter the nozzles due to the vacuum created by the movement of the liquid in the capillary tube resulting from acceleration/deceleration of the cartridge. Alternatively, an air bubble from the cartridge may travel up through the capillary tube and block the die from releasing fluid composition through a nozzle on the die.

As such, it would be beneficial to provide a cartridge having a microfluidic delivery member that is configured to release a liquid composition up into the air while minimizing opportunities for air bubbles to get trapped in the die.

### SUMMARY

Aspects of the present disclosure include a cartridge for a microfluidic delivery system. The cartridge includes a reservoir for containing a fluid composition. The cartridge includes a capillary tube having a first end portion and a second end portion opposing the first end portion. The first end portion of the capillary tube is in fluid communication with the reservoir. The second end portion of the capillary tube is operatively connected with the reservoir. The cartridge includes a restriction member in fluid communication with the capillary tube and configured to restrict fluid flow. The cartridge includes a microfluidic delivery member connected with and in fluid communication with the reservoir. The microfluidic delivery member comprises a die having at least one nozzle.

Aspects of the present disclosure also include a microfluidic delivery system. The microfluidic delivery system includes a housing having a base, at least one sidewall connected with the base, and an opening for receiving a cartridge at least partially within the housing. The microfluidic delivery system includes a cartridge releasably and electrically connectable with the housing. The cartridge comprises a reservoir containing a fluid composition to be dispensed from at least one nozzle. The cartridge also comprises a capillary tube having a first end portion and a second end portion opposing the first end portion. The first end portion of the capillary tube is in fluid communication with the reservoir and the second end portion of the capillary tube is operatively connected with the reservoir. The cartridge includes a restriction member in fluid communication within the capillary tube and configured to restrict fluid flow.

Aspects of the present disclosure include a method of delivering a composition into the air. The method comprises the steps of: providing a microfluidic delivery device, the microfluidic delivery device comprising a reservoir, a capillary tube in fluid communication with the reservoir, and a microfluidic delivery member in fluid communication with the capillary tube and connected with the reservoir, wherein the reservoir contains a fluid composition; directing the fluid composition from the reservoir, through the capillary tube, and to the microfluidic deliver member; restricting the flow of the fluid composition to a first flow rate as the fluid composition flows through a portion of the capillary tube; and atomizing the fluid composition into the air at a second flow rate, wherein second flow rate is at least about 1.5 times greater than the first flow rate.

### BRIEF DESCRIPTION OF THE DRAWINGS

FIG. 1 is a perspective view of a microfluidic delivery system including a housing having a cartridge disposed therein and a charger for recharging rechargeable batteries used to power the microfluidic delivery system.

FIG. 2 is a perspective view of the housing of the microfluidic delivery system of FIG. 1 without a charger or cartridge connected therewith.

FIG. 3 is a sectional view of FIG. 2 taken along line 3-3.

FIG. 4 is a bottom, plan view of the housing of FIG. 2.

FIG. 5 is a schematic, perspective view of a housing having a cartridge disposed therein, and comprising a door for accessing the interior of the housing.

FIG. 6 is a perspective view of a cartridge having a reservoir and an outer cover.

FIG. 7 is a sectional view of FIG. 6 taken along line 7-7.

FIG. 8 is a sectional view of FIG. 6 taken along line 8-8.

FIG. 9 is a schematic, side elevation view of a capillary tube.

FIG. 10 is a schematic, sectional view of FIG. 9 taken along line 10-10.

FIG. 11 is a schematic, sectional view of FIG. 9 taken along line 11-11.

FIG. 12 is a schematic, sectional view of an exemplary capillary tube.

FIG. 13 is a schematic, sectional view of FIG. 9 taken along line 13-13.

FIG. 14 is a schematic, perspective view of an exemplary restriction member for a capillary tube.

FIG. 15A is a schematic, elevation view of a capillary tube having a restriction member in the form of an orifice plate.

FIG. 15B is a schematic, perspective view of an exemplary restriction member in the form of an orifice plate.

FIG. 15C is a view of FIG. 15A taken along line 15C-15C.

FIG. 16 is an enlarged view of portion 16 of FIG. 7.

FIG. 17A is a top, perspective view of a microfluidic delivery member having a rigid PCB.

FIG. 17B is a bottom, perspective view of a microfluidic delivery member having a rigid PCB.

FIG. 18A is a perspective view of a semi-flex PCB for a microfluidic delivery member.

FIG. 18B is a side, elevation view of a semi-flex PCB for a microfluidic delivery member.

FIG. 19 is an exploded view of a microfluidic delivery member.

FIG. 20 is a top, perspective view of a die of a microfluidic delivery member.

FIG. 21 is a top, perspective view of a die with a nozzle plate removed to show fluid chambers of the die.

FIG. 22 is a top, perspective view of a die with layers of the die removed to show the dielectric layer of the die.

### DETAILED DESCRIPTION

The present disclosure provides a microfluidic delivery system comprising cartridge having a microfluidic delivery member and methods for delivering fluid compositions into the air.

The microfluidic delivery system of the present disclosure may include a housing and a cartridge. The cartridge may be fixed with the housing, removably connectable with the housing, and/or replaceable, and may be disposed at least partially within the housing. The cartridge may comprise a reservoir for containing a volatile composition, a microfluidic delivery member, and a capillary tube disposed within the reservoir and configured to deliver a fluid composition from within the reservoir to the microfluidic delivery member. The microfluidic delivery member may be configured to dispense the fluid composition into the air. The cartridge is electrically connectable with the housing.

The cartridge may include a capillary tube disposed within an interior of the reservoir. The capillary tube may be defined by a first end portion, a second end portion, and a central portion. The capillary tube may be defined by an interior and an exterior. The first end portion may be arranged in fluid communication with the fluid composition in the reservoir and the second end portion may be operatively connected with the reservoir. The second end of the capillary tube is located below the microfluidic delivery member. The capillary tube delivers fluid composition from the reservoir to the microfluidic delivery member. Fluid composition can travel by capillary action, suction, siphon, vacuum, or other mechanism.

The capillary tube may include a restriction member. The restriction member may be in fluid communication with the capillary tube. The restriction member may be disposed at least partially within the interior of the capillary tube.

The restriction member may be configured in various ways. For example, the restriction member may be integral with the capillary tube. Or, the restriction member may be configured as a separate component that is mechanically connected with the capillary tube.

The restriction member may comprise a porous material. The restriction member may be configured as an orifice plate.

While the below description describes the microfluidic delivery system comprising a housing and a cartridge, both having various components, it is to be understood that the microfluidic delivery system is not limited to the construction and arrangement set forth in the following description or illustrated in the drawings. The microfluidic delivery system and cartridge of the present disclosure are applicable to other configurations or may be practiced or carried out in various ways. For example, the components of the housing may be located on the cartridge and vice-versa. Further, the housing and cartridge may be configured as a single unit versus constructing a cartridge that is separable from the housing as described in the following description. Moreover, the cartridge may be used with various devices for delivering fluid composition into the air or onto a target surface.

### Housing

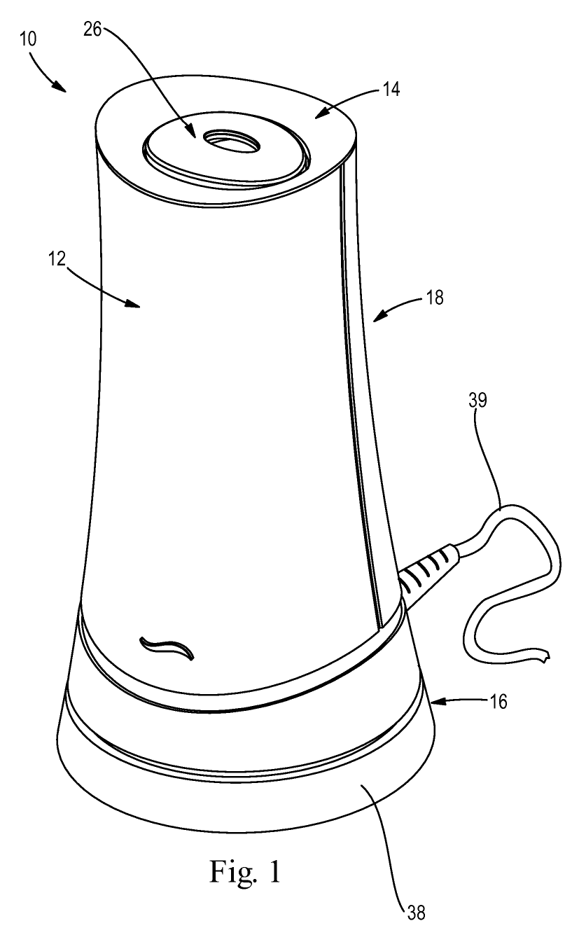

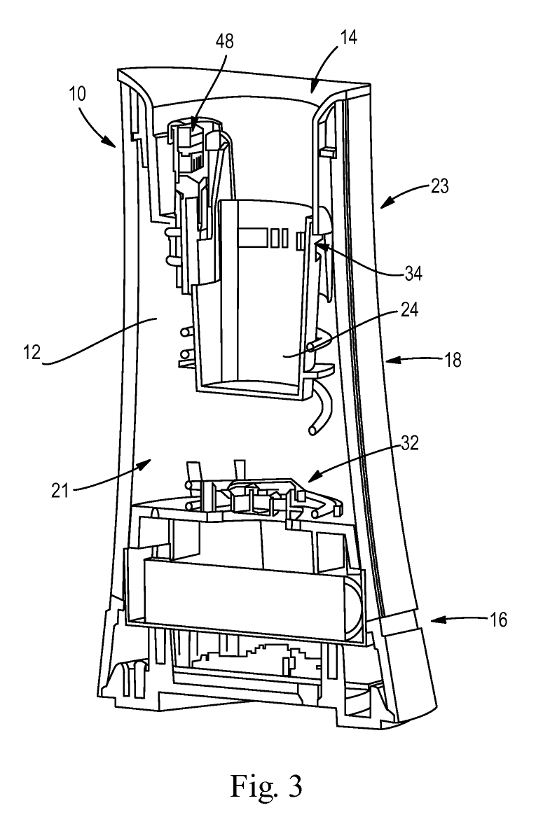

With reference to FIGS. 1-3, the microfluidic delivery system 10 may include a housing 12. The housing 12 may be constructed from a single component or have multiple components that are combined to form the housing 12. The housing 12 may be defined by an interior 21 and an exterior 23. The housing 12 may be comprised of an upper portion 14, a lower portion 16, and a body portion 18 that extends between and connects the upper portion 14 and the lower portion 16.

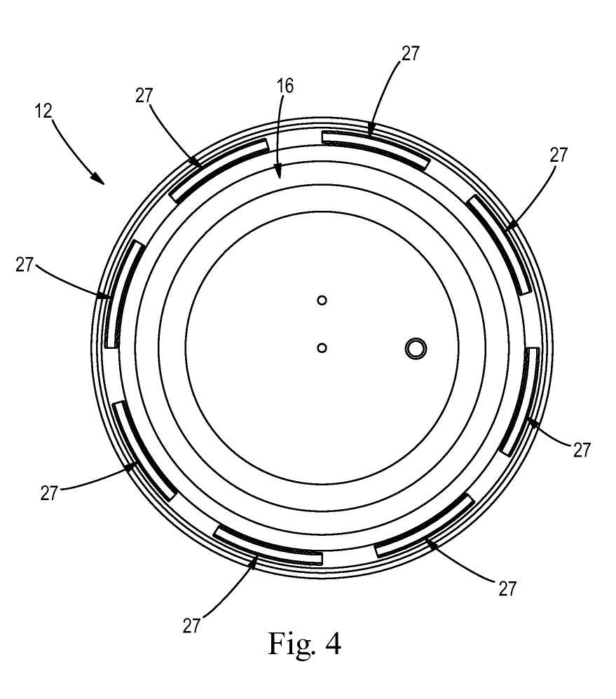

The housing 12 may include an opening 20 in the upper portion 14 of the housing 12 and a holder 24 for receiving and holding the cartridge 26 in the housing 12. The cartridge 26 may be received into the upper portion 14 of the housing 12. An air flow channel 34 may be formed between the holder 24 and the upper portion 14 of the housing 12. With reference to FIG. 4, the housing 12 may comprise one or more air inlets 27. The air inlets 27 may be positioned in the lower portion 16 of the housing, as shown in FIG. 4 for illustrative purposes only, or may be formed in the body portion 18 of the housing.

The microfluidic delivery system 10 may comprise a fan 32 to assist in driving room-fill and/or to help prevent deposition of larger droplets from landing on surfaces adjacent to the microfluidic delivery system 10 that could damage the surface. The fan 32, for example, may be disposed at least partially within the interior 21 of the housing 12 and may be positioned between the holder 24 and the lower portion 16 of the housing 12. However, the fan may be configured and arranged in any other way suitable for the desired use. An exemplary fan includes a 5V 25×25×8 mm DC axial fan (Series 250, Type255N from EBMPAPST), that is capable of delivering about 10 to about 50 liters of air per minute (l/min), or about 15 l/min to about 25 l/min. As will be discussed in more detail below, the fan 32 pulls air from the air inlet(s) 27 into the housing 12 and directs the air up through the air flow channel 34 toward the cartridge 26. The air velocity exiting the opening 20 may be in the range of about 1 meter per second (m/s) to about 5 m/s, or about 1.5 m/s to about 2.5 m/s.

The microfluidic delivery system 10 may be in electrical communication with a power source. The power source may be located in the interior 21 of the housing 12, such as a disposable battery or a rechargeable battery. Or, the power source may be an external power source such as an electrical outlet that connects with a power cord 39 connected with the housing 12. The housing 12 may include an electrical plug that is connectable with an electrical outlet. The microfluidic delivery system may be configured to be compact and easily portable. As such, the power source may include rechargeable or disposable batteries. The microfluidic delivery system may be capable for use with electrical sources as 9-volt batteries, conventional dry cells such as “A”, “AA”, “AAA”, “C”, and “D” cells, button cells, watch batteries, solar cells, as well as rechargeable batteries with recharging base.

With reference to FIG. 1, the microfluidic delivery system 10 may be powered by rechargeable batteries disposed within the interior 21 of the housing. The rechargeable batteries may be charged using a charger 38. The charger 38 may include an power cord 39 that connects with an external power source, such as an electrical outlet or battery terminals. The charger 38 may receive the housing 12 to charge the batteries. As will be discussed in more detail below, electrical contacts 48 disposed on the interior 21 of the housing couple with the internal or external power source and couple with electrical contacts on the microfluidic delivery member of the cartridge to power the die. The housing 12 may include a power switch on exterior 23 of the housing 12.

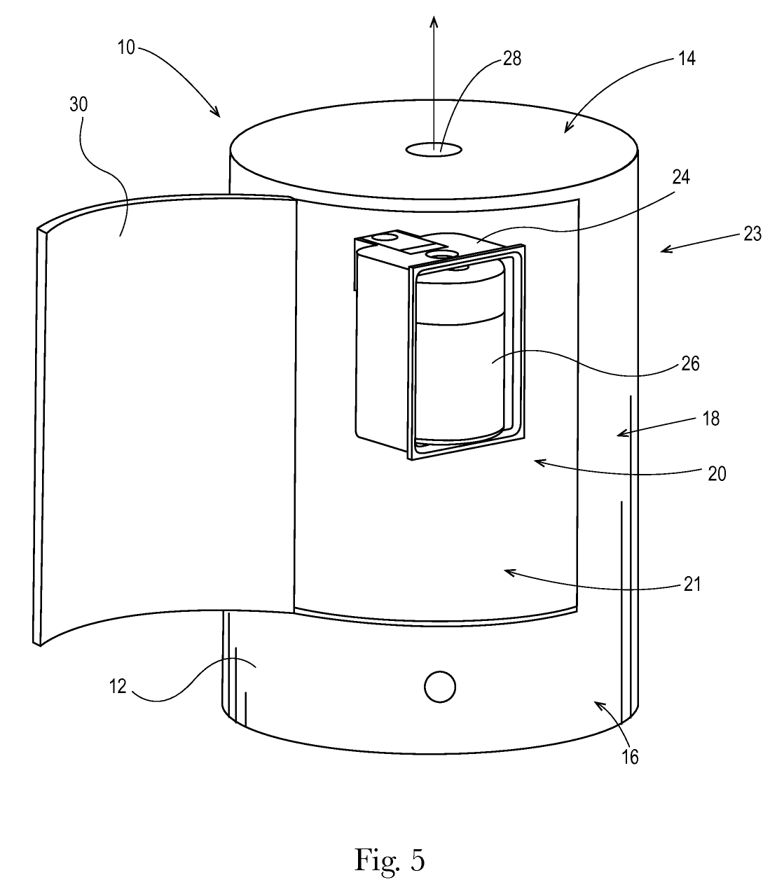

With reference to FIG. 5, the opening 20 may be disposed in the upper or body portion 14 or 18 of the housing 12. The housing 12 may include a door 30 or structure to cover the opening 20. The cartridge 26 may slide in through the opening in the body portion 18 of the housing 12. The housing 12 may include air outlet 28 that places an environment on the exterior 23 of the housing 12 in fluid communication with the interior 21 of the housing 12. The door 30 may rotate to provide access to the air outlet 28. However, it is to be appreciated that the door or covering may be configured in various different ways. The door 30 may form a substantially air tight connection with the remainder of the housing 12 such that pressurized air in the interior 21 of the housing 12 does not escape through any gaps between the door 30 and the housing.

### Cartridge

With reference to FIGS. 1 and 6-8, the cartridge 26 may have a longitudinal axis A and may comprise a reservoir 50 for containing a fluid composition 52. The cartridge 26 may include a die 92 and a capillary tube 80. The capillary tube 80 may be configured to deliver fluid composition from the reservoir 50 to the die 92. The die 92 may be configured to dispense the fluid composition into the air or onto a target surface. Fluid may travel from the reservoir 50 into the capillary tube 80 through capillary action, or by vacuum. As described in more detail below, the capillary tube 80 may include a restriction member 81. The restriction member 81 is configured to restrict fluid flow through a portion of the capillary tube 80 in order to prevent air bubbles from the environment or from the reservoir 50 from blocking fluid flow through the microfluidic delivery member 64.

### Reservoir

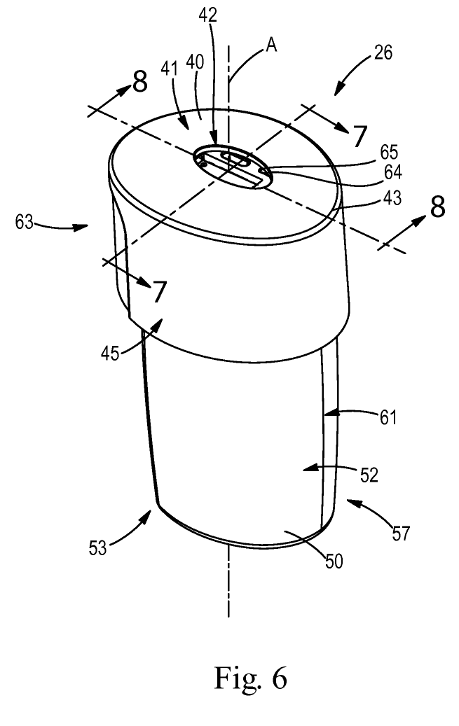

With reference to FIGS. 6-8, the cartridge 26 includes a reservoir 50 for containing a fluid composition. The reservoir 50 may be configured to contain from about 5 milliliters (mL) to about 100 mL, alternatively from about 10 mL to about 50 mL, alternatively from about 15 mL to about 30 mL of fluid composition. The cartridge 26 may be configured to have multiple reservoirs, with each reservoir containing the same or a different fluid composition. The reservoir can be made of any suitable material for containing a fluid composition including glass, plastic, metal, or the like.

The reservoir 50 may be comprised of a top portion 51, a base portion 53 opposing the top portion 51, and at least one sidewall 61 connected with and extending between the top portion 51 and the base portion 53. The reservoir 50 may define an interior 59 and an exterior 57. The top portion 51 of the reservoir 50 may include an air vent 93 and a fluid outlet 90. While the reservoir 50 is shown as having a top portion 51, a base portion 53, and at least one sidewall 61, it is to be appreciated that the reservoir 50 may be configured in various different ways.

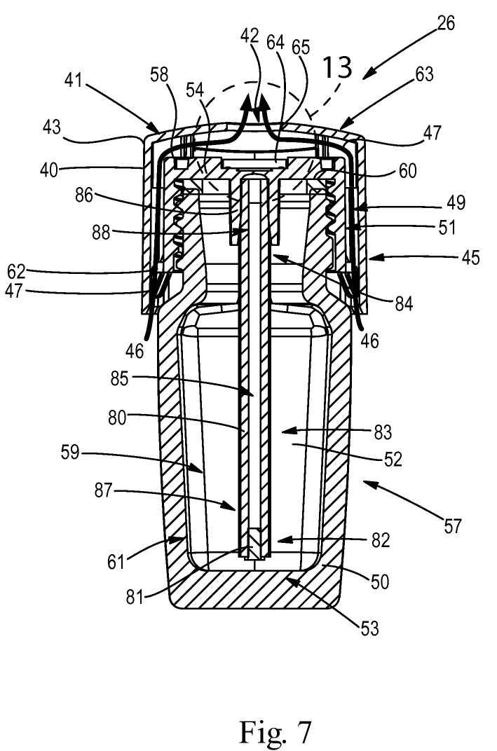

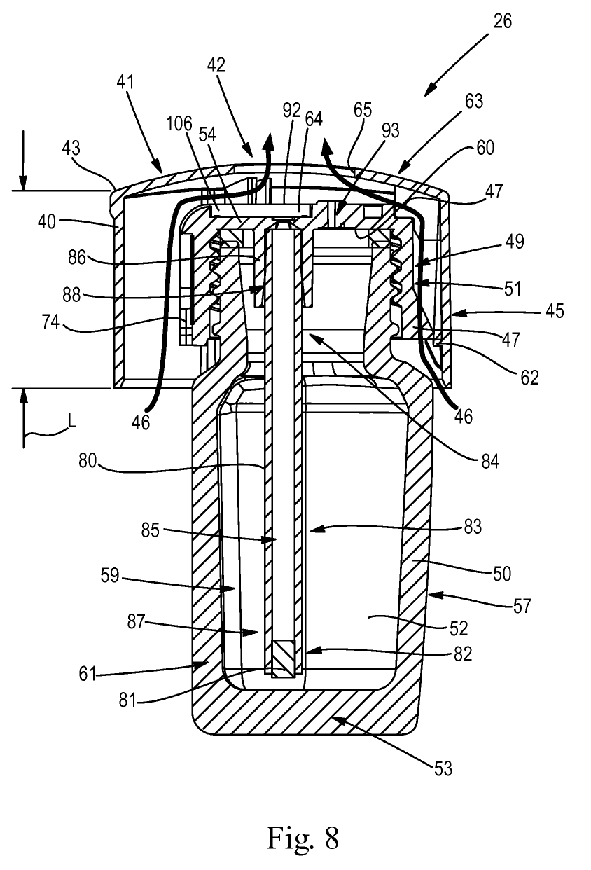

The reservoir 50, including the top portion 51, base portion 53, and sidewall(s) 61, may be configured as a single element or may be configured as separate elements that are joined together. For example, the top portion 51 or base portion 53 may be configured as a separate element from the remainder of the reservoir 50. For example, with reference to FIGS. 7 and 8, the reservoir 50 may be comprised of two elements joined together; the base portion 53 and the sidewall(s) 61 may be one element and the top portion 51 may be a separate element. The top portion 51 may be configured as a lid 54 that is mechanically connected with the sidewall(s) 61. The lid 54 may be removably or fixably connected with the sidewall(s) 61 to substantially enclose the reservoir 50. The lid 54 may be threadingly attached with the sidewall(s) 61 of the reservoir 50, or may be welded, glued, or the like with the sidewall(s) 61 of the reservoir 50.

With reference to FIGS. 7-8, the reservoir 50 may include a connection member 86 extending from the interior 59 of the reservoir 50. The connection member 86 may define a chamber 88 for receiving a portion of the second end portion 84 of the capillary tube 80. The chamber 88 may be substantially sealed between the connection member 86 and the capillary tube 80 to prevent air from the reservoir 50 from entering the chamber 88.

In an example configuration wherein the top portion 51 of the reservoir 50 includes a lid 54, the connection member 86 may extend from the lid 54. The lid 54 of the reservoir may be defined by an outer surface 58 and an inner surface 60. The lid 54 may include a connection member 86 extending from the inner surface 60.

The reservoir may be transparent, translucent, or opaque or any combination thereof. For example, the reservoir may be opaque with a transparent indicator of the level of fluid composition in the reservoir.

### Capillary Tube

With reference to FIGS. 7-10, the cartridge 26 includes a capillary tube 80 disposed within the interior 59 of the reservoir 50. The capillary tube 80 may be defined by a first end portion 82, a second end portion 84, and a central portion 83. The capillary tube 80 may be defined by an interior 85 and an exterior 87. The first end portion 82 is in fluid communication with a fluid composition 52 in the reservoir 50 and the second end portion 84 is operatively connected with the connection member 86 of the reservoir 50. The second end 84 of the capillary tube 80 is located below the microfluidic delivery member 64. The capillary tube 80 delivers fluid composition from the reservoir 50 to the microfluidic delivery member 64. Fluid composition can travel by capillary action, suction, siphon, vacuum, or other mechanism against the force of gravity.

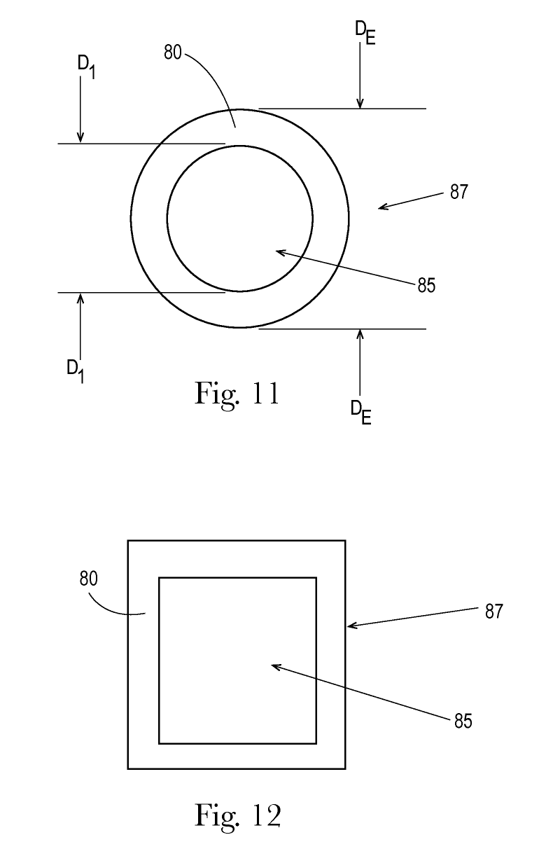

The capillary tube 80 may be any shape that is able to deliver fluid from the reservoir 50 to the microfluidic delivery member 64. With reference to FIGS. 7-11, the capillary tube 80 may have a cylindrical shape. However, the capillary tube 80 may have various shaped cross-sections. For example, the cross-section of the capillary tube 80 may be circular, such as shown in FIG. 11 for illustrative purposes only; square, such as shown in FIG. 12 for illustrative purposes only; rectangular; or arcuate shaped.

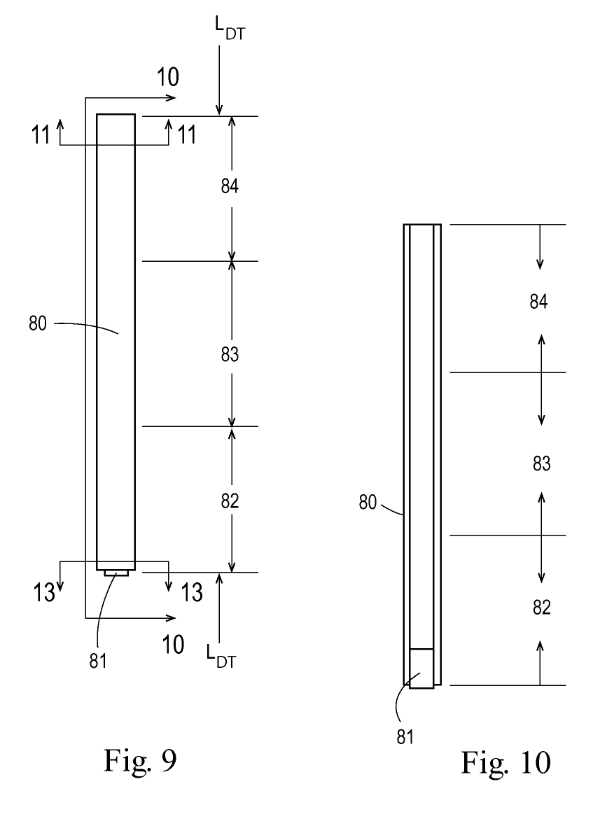

The capillary tube 80 may have various dimensions. The capillary tube 80 may be defined by a length L_{DT} that extends from one end of the capillary tube 80 to the opposite end of the capillary tube 80. The capillary tube 80 can be various lengths L_{DT}. For example, with reference to FIG. 9, the length L_{DT} of the capillary tube 80 may be from about 1 mm to about 100 mm, or from about 5 mm to about 75 mm, or from about 10 mm to about 50 mm. The interior 85 of the capillary tube 80 may be defined by a diameter D_{I}.

With reference to FIG. 11, the diameter D_{I} of the interior 85 of the capillary tube 80 may be various dimensions. For example, the diameter D_{I} of the interior 85 of the capillary tube 80 may be from about 0.5 mm to about 5 mm, or from about 0.5 mm to about 3 mm, or from about 0.5 mm to about 1.5 mm. The exterior 87 of the capillary tube 80 may be defined by a diameter D_{E}. The diameter D_{E} of the exterior 87 of the capillary tube 80 may be various dimensions. For example, the diameter D_{E} of the exterior 87 of the capillary tube 80 may be from about 0.5 mm to about 10 mm, or from about 1 mm to about 5 mm, or from about 1 mm to about 3 mm.

The capillary tube 80 may be comprised of various materials. For example, the capillary tube 80 may be comprised of glass, a rigid or semi-rigid plastic material, or the like.

As discussed above, and with reference to FIGS. 7-10 and 13, the capillary tube 80 may include a restriction member 81. The restriction member 81 may be in fluid communication with the capillary tube 80. The restriction member 81 may be disposed at least partially within the interior 85 of the capillary tube 80. The restriction member 81 may be configured to restrict fluid flow through a portion of the capillary tube 80, especially during an event such as a sudden acceleration or deceleration of the cartridge. It may be advantageous to configure the restriction member 81 such that it provides a large restriction to flow (pressure drop) during an acceleration/deceleration event where the instantaneous pressure applied across the restriction member is very high. At the same time, it may be desirable that the restriction element provides an insignificant restriction to flow during normal dispensing.

The restriction member 81 may prevent air from within the reservoir 50 from traveling up through the capillary tube 80 and into the die 92 of the microfluidic delivery member 64. An example of such an event might be when the cartridge reservoir is tilted such that the liquid-air free surface of the liquid drops below the first end portion 82 of the capillary tube 80. In this case, the restriction member 81 maintains the presence of liquid in the capillary tube, and prevents the entry of air into said first end portion 82. If air bubbles make it into the die 92, the air bubbles can get trapped in a nozzle(s) 130 and prevent fluid 52 from being released from the die 92. This, in turn, can reduce the rate that fluid is released from the cartridge 26 into the air.

The restriction member 81 is arranged in fluid communication with the capillary tube to prevent air from entering the die. For example, with reference to FIGS. 9, 10, and 15A, the restriction member 81 may be arranged in various locations relative to the capillary tube 80. For example, the restriction member 81 may be connected with the first end portion 82 of the capillary tube 80. The restriction member 81 may be disposed at least partially within the first end portion 82 of the capillary tube 80. The restriction member 81 may be partially disposed within the interior 85 capillary tube 80. Or, the restriction member 81 may be completely disposed within the interior 85 of the capillary tube 80. While the restriction member 81 is shown in FIGS. 7 and 8 as being partially disposed within the first end portion 82 of the capillary tube 80, it is to be appreciated that the restriction member 81 may be disposed in the first end portion 82, the second end portion 84, and/or the central portion 83. Positioning the restriction member 81 in the first end portion 82 of the capillary tube 80 may be more advantageous than positioning in other portions in preventing an air bubble from entering an air bubble from even entering the capillary tube 80.

With reference back to FIGS. 7 and 8, the restriction member 81 may be configured in various ways. For example, the restriction member 81 may be integral with the capillary tube 80. Or, the restriction member 81 may be configured as a separate component that is mechanically connected with the capillary tube 80.

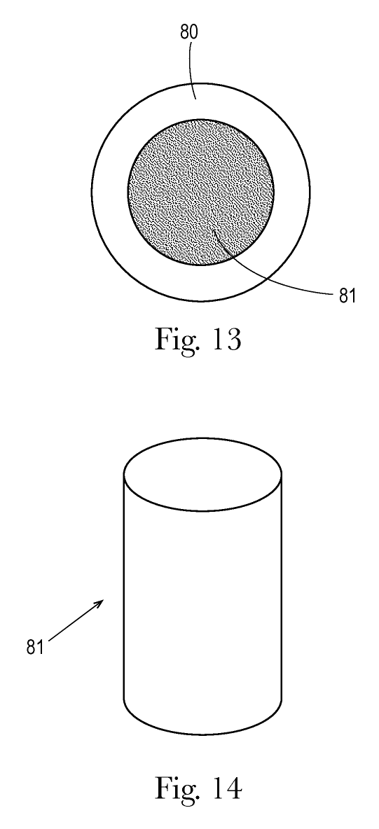

With reference to FIGS. 13 and 14, the restriction member 81 may comprise a porous material. For example, the restriction member 81 may be configured as an open cell foam, fibrous wick, porous plastic wick, sponge or the like. The porous material may be made of various materials, including fabrics of nylon, cotton, fiber glass, and plastics such as polyethylene, ultra-high molecular weight polyethelene, nylon 6, polypropylene, polyester fibers, ethyl vinyl acetate, polyether sulfone, polyvinylidene fluoride, polyethersulfone, polytetrafluroethylene, combinations thereof, and the like.

If the restriction member 81 is configured as a wick, the wick may be a high density wick. The wick may have a pore radius or equivalent pore radius (e.g. in the case of fiber based wicks) ranging from about 20 microns to about 200 microns, alternatively from about 30 microns to about 150 microns, alternatively from about 30 microns to about 125 microns, alternatively, about 40 microns to about 100 microns.

Regardless of the material of manufacture, where a porous material is used for the restriction member 81, the restriction member 81 can exhibit an average pore size from about 10 microns to about 500 microns, alternatively from about 50 microns to about 150 microns, or alternatively about 70 microns. The average pore volume of the restriction member 81, expressed as a fraction of the restriction member 81 not occupied by the structural composition, is from about 15% to about 85%, alternatively from about 25% to about 50%.

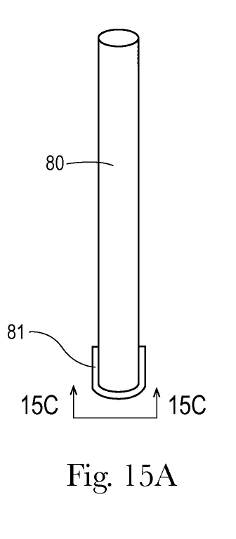

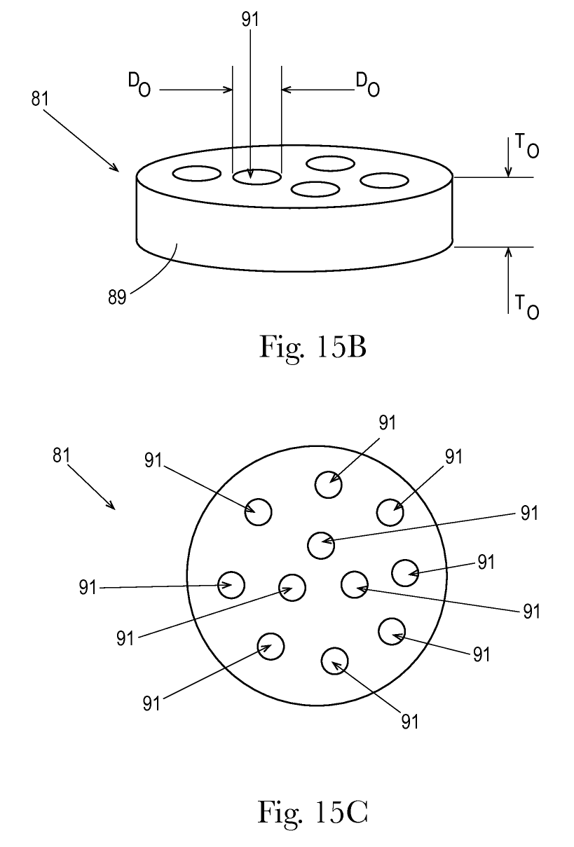

With reference to FIGS. 15A-15C, the restriction member 81 may be configured as an orifice plate 89. The orifice plate 89 may have various shapes and may connect with the capillary tube 80 in various ways. For example, the restriction member 81 may be connected with the capillary tube 80 at the first end portion 82. The restriction member 81 may fit over the end of the second end portion 82 of the capillary tube. Or, the restriction member 81 may be partially disposed within the end of the second end portion 82 of the capillary tube. The restriction member 81 may also be disposed completely within the first, second, and/or central portions 82, 84, and/or 83 of the capillary tube 80.

The orifice plate may have a cylindrical shape. The orifice plate 89 may have one or more orifices 91 that are defined by a diameter D_{O} that allow fluid to pass through the orifice plate 89. The orifice(s) 91 may have a diameter D_{O} of about 20 μm to about 1 mm. The orifice plate may have a thickness T_{O} in the range of about 0.5 mm to about 3 mm. The orifice plate 89 may be comprised of various materials, including glass or various polymer materials. The orifice plate 89 may be composed of the same material as the capillary tube or may be composed of a material that is different from the material of the capillary tube.

The restriction member 81 is configured to restrict the flow of fluid through a portion of the capillary tube 80. For example, the flow rate of fluid passing through the restriction member 81 may be defined by a first flow rate and the flow rate of fluid exiting the microfluidic die 92 of the cartridge 26 may be defined by a second flow rate. The second flow rate may be greater than the first flow rate. The second flow rate may be at least about 1.5 times, or at least about 2 times greater than the first flow rate.

The back pressure applied to the fluid composition at the restriction member 81 may be in the range of about 249 pascal (Pa) to about 10 kPa, or about 249 Pa to about 5 kPa, or about 249 Pa to about 2.5 kPa. The fluid pressure at the restriction member 81 may be in the range of about 249 Pa to about 2.5 kPa, or about 249 Pa to about 1.25 kPa, or about 249 Pa to about 0.75 kPa. The fluid pressure at the die may be in the range of about 20 kPa to about 25 kPa.

### Microfluidic Delivery Member

With reference to FIGS. 7-8 and 16-18B, the microfluidic delivery system 10 may comprise a microfluidic delivery member 64 that utilizes aspects of ink-jet print head systems, and more particularly, aspects of thermal or piezo ink-jet print heads. The microfluidic delivery member 64 may be connected with the top portion 51 and/or sidewall 61 of the reservoir 50 of the cartridge 26.

In a “drop-on-demand” ink-jet printing process, a fluid composition is ejected through a very small orifice of a diameter typically about 5-50 microns, or between about 10 and about 40 microns, in the form of minute droplets by rapid pressure impulses. The rapid pressure impulses are typically generated in the print head by either expansion of a piezoelectric crystal vibrating at a high frequency or volatilization of a volatile composition (e.g. solvent, water, propellant) within the ink by rapid heating cycles. Thermal ink-jet printers employ a heating element within the print head to volatilize a portion of the composition that propels a second portion of fluid composition through the orifice nozzle to form droplets in proportion to the number of on/off cycles for the heating element. The fluid composition is forced out of the nozzle when needed. Conventional ink-jet printers are more particularly described in U.S. Pat. Nos. 3,465,350 and 3,465,351.

The microfluidic delivery member 64 may be in electrical communication with a power source and may include a printed circuit board (“PCB”) 106 and a die 92 that is in fluid communication with the capillary tube 80.

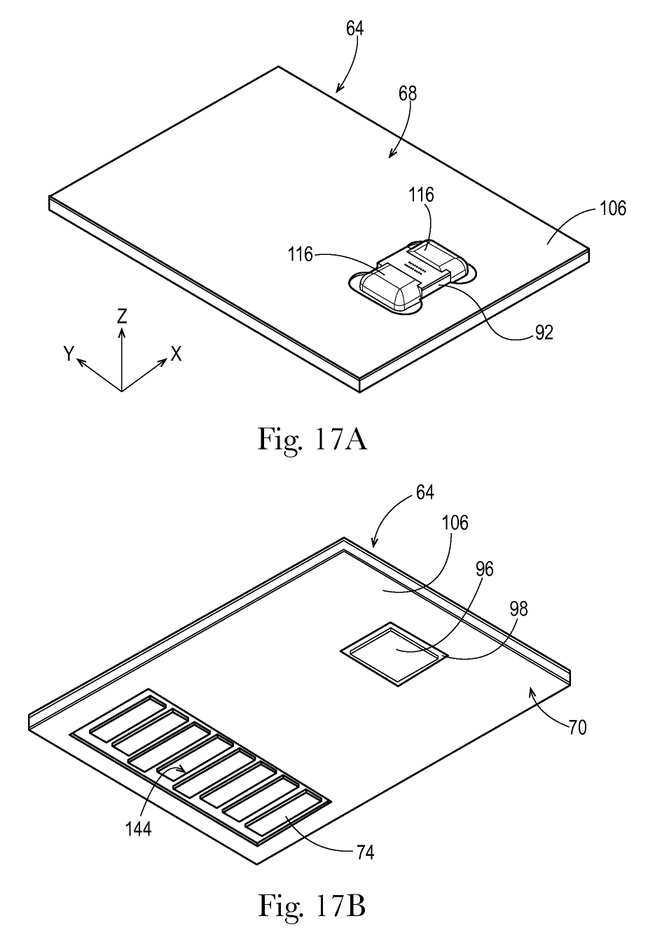

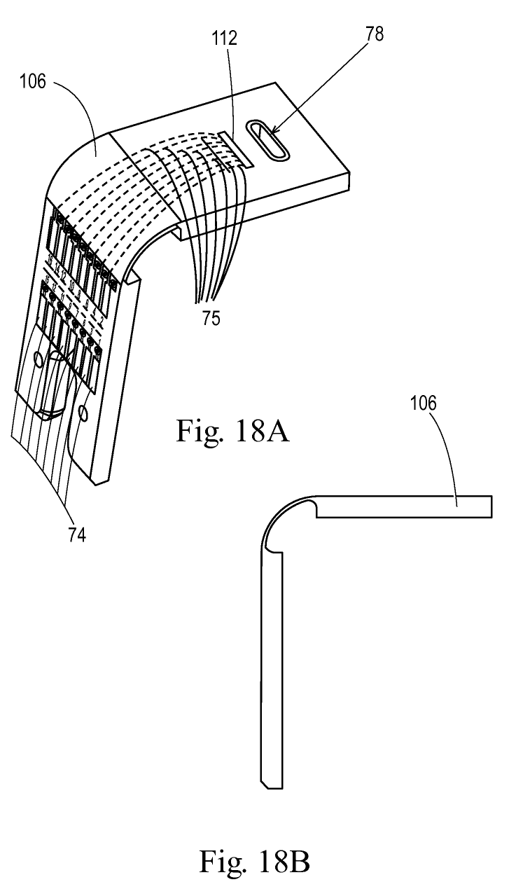

The PCB 106 may be a rigid planar circuit board, such as shown in FIGS. 17A and 17B for illustrative purposes only; a flexible PCB; or a semi-flex PCB, such as shown in FIGS. 18A and 18B for illustrative purposes only. The semi-flex PCB shown in FIGS. 18A and 18B may include a fiberglass-epoxy composite that is partially milled in a portion that allows a portion of the PCB 106 to bend. The milled portion may be milled to a thickness of about 0.2 millimeters. The PCB 106 has upper and lower surfaces 68 and 70.

The PCB 106 may be of a conventional construction. It may comprise a ceramic substrate. It may comprise a fiberglass-epoxy composite substrate material and layers of conductive metal, normally copper, on the top and bottom surfaces. The conductive layers are arranged into conductive paths through an etching process. The conductive paths are protected from mechanical damage and other environmental effects in most areas of the board by a photo-curable polymer layer, often referred to as a soldermask layer. In selected areas, such as the liquid flow paths and wire bond attachment pads, the conductive copper paths are protected by an inert metal layer such as gold. Other material choices could be tin, silver, or other low reactivity, high conductivity metals.

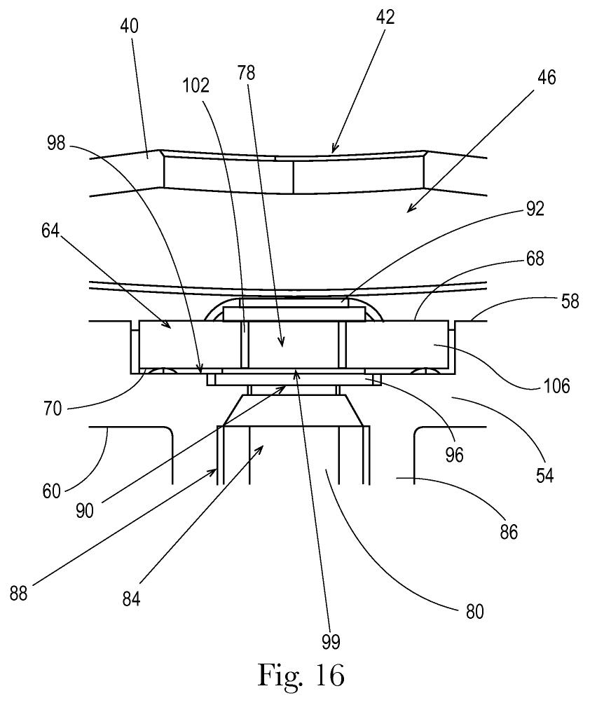

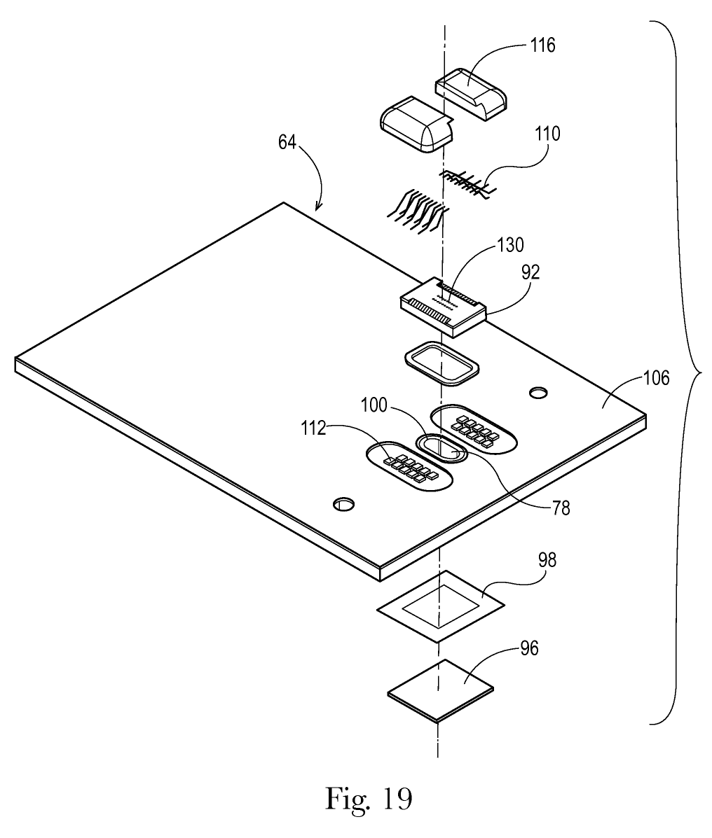

Still referring to FIGS. 16-18B, the PCB 106 may include all electrical connections—the contacts 74, the traces 75, and the contact pads 112. The contacts 74 and contact pads 112 may be disposed on the same side of the PCB 106, or may be disposed on different sides of the PCB. For example, as shown in FIGS. 17A, 17B, and 19, the contacts 74 may be disposed on opposite sides of the PCB 106. The contacts 74 may be disposed on the lower surface 70 of the PCB 106 and the contact pads 112 may be disposed on the upper surface 68 of the PCB 106.

With reference to FIGS. 17A and 17B, the die 92 and the contacts 74 may be disposed on parallel planes. This allows for a simple, rigid PCB 106 construction. The contacts 74 and the die 92 may be disposed on the same side of the PCB 106 or may be disposed on opposite sides of the PCB 106. In such a configuration, the contacts 74 and the die 92 may be disposed on the upper surface 68.

With reference to FIGS. 18A and 18B, the contacts 74 may be disposed on the same side as the contact pads 112. For example, the contacts 74 and the contact pads 112 may be disposed on the upper surface 68.

With reference to FIGS. 16, 17B, and 19, the microfluidic delivery member 64 may include a filter 96. The filter 96 may be disposed on the lower surface 70 of the PCB 106. The filter 96 may separate the opening 78 of the board from the chamber 88 at the lower surface of the board. The filter 96 may be configured to prevent at least some particulates from the fluid composition from passing through the opening 78 and clogging the nozzles 130 of the die 92. The filter 96 may be configured to block particulates that are greater than one third of the diameter of the nozzles 130. It is to be appreciated that the capillary tube 80 can act as a suitable filter 96, so that a separate filter is not needed. The filter 96 may be a stainless steel mesh. The filter 96 may be randomly weaved mesh, polypropylene or silicon based.

The filter 96 may be attached to the bottom surface 70 with an adhesive material that is not readily degraded by the fluid composition in the reservoir 50. The adhesive may be thermally or ultraviolet activated. The filter 96 is positioned between the chamber 88 and the die 92. The filter 96 is separated from the bottom surface 70 of the microfluidic delivery member 64 by a mechanical spacer 98. The mechanical spacer 98 creates a gap 99 between the bottom surface 70 of the microfluidic delivery member 64 and the filter 96 proximate the opening 78. The mechanical spacer 98 may be a rigid support or an adhesive that conforms to a shape between the filter 96 and the microfluidic delivery member 64. In that regard, the outlet of the filter 96 is greater than the diameter of the opening 78 and is offset therefrom so that a greater surface area of the filter 96 can filter fluid composition than would be provided if the filter was attached directly to the bottom surface 70 of the microfluidic delivery member 64 without the mechanical spacer 98. It is to be appreciated that the mechanical spacer 98 allows suitable flow rates through the filter 96. That is, as the filter 96 accumulates particles, the filter 96 will not slow down the fluid flowing therethrough. The outlet of the filter 96 may be about 4 mm² or larger and the standoff is about 700 microns thick.

The opening 78 may be formed as an oval, as is illustrated in FIG. 16; however, other shapes are contemplated depending on the application. The oval may have the dimensions of a first diameter of about 1.5 mm and a second diameter of about 700 microns. The opening 78 exposes sidewalls 102 of the PCB 106. If the PCB 106 is an FR4 PCB, the bundles of fibers would be exposed by the opening. These sidewalls are susceptible to fluid composition and thus a liner 100 is included to cover and protect these sidewalls. If fluid composition enters the sidewalls, the PCB 106 could begin to deteriorate, cutting short the life span of this product.

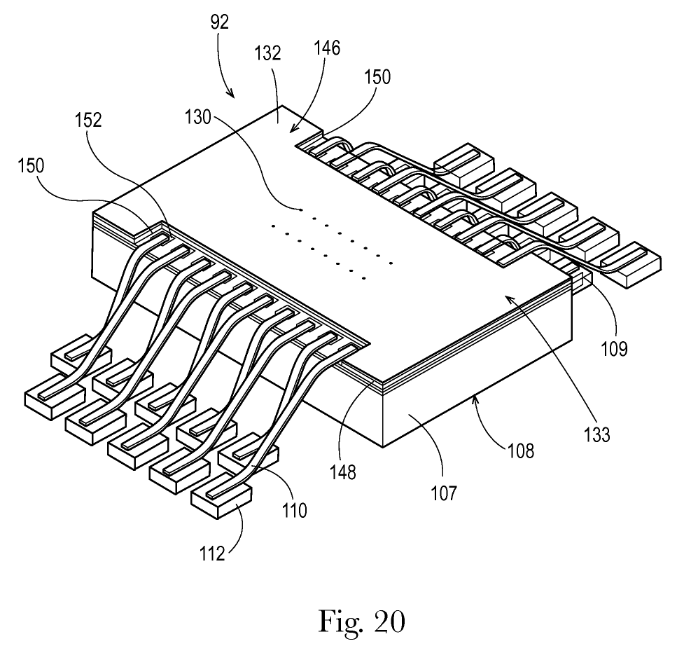

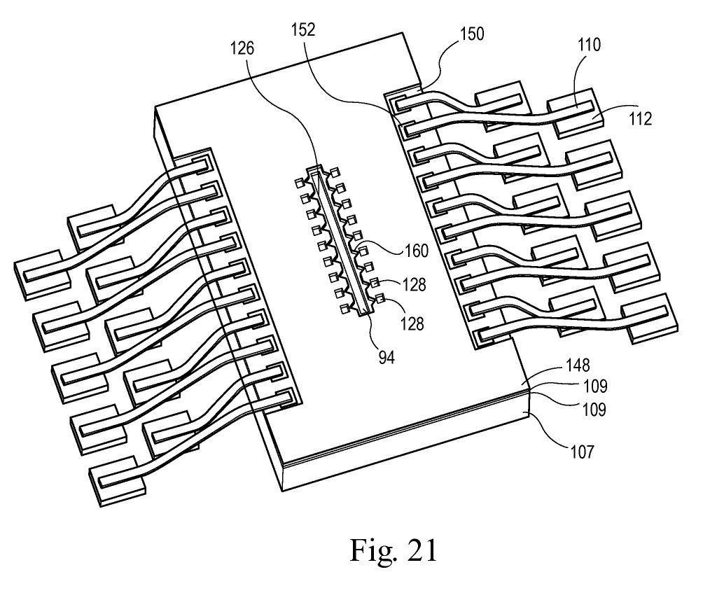

With reference to FIGS. 20 and 21, the PCB 106 may carry a die 92. The die 92 comprises a fluid injection system made by using a semiconductor micro fabrication process such as thin-film deposition, passivation, etching, spinning, sputtering, masking, epitaxy growth, wafer/wafer bonding, micro thin-film lamination, curing, dicing, etc. These processes are known in the art to make MEMs devices. The die 92 may be made from silicon, glass, or a mixture thereof. The die 92 comprises a plurality of microfluidic chambers 128, each comprising a corresponding actuation element: heating element or electromechanical actuator. In this way, the die's fluid injection system may be micro thermal nucleation (e.g. heating element) or micro mechanical actuation (e.g. thin-film piezoelectric). One type of die for the microfluidic delivery member is an integrated membrane of nozzles obtained via MEMs technology as described in U.S. 2010/0154790, assigned to STMicroelectronics S.R.I., Geneva, Switzerland. In the case of a thin-film piezo, the piezoelectric material (e.g. lead zirconinum titanate)” is typically applied via spinning and/or sputtering processes. The semiconductor micro fabrication process allows one to simultaneously make one or thousands of MEMS devices in one batch process (a batch process comprises of multiple mask layers).

With reference to FIG. 19, the die 92 may be secured to the upper surface 68 of the PCB 106 above the opening 78. The die 92 may be secured to the upper surface of the PCB 106 by any adhesive material configured to hold the semiconductor die to the board. The adhesive material may be the same or different from the adhesive material used to secure the filter 96 to the microfluidic delivery member 64.

With reference to FIGS. 17A-19, the PCB 106 includes the electrical contacts 74 at the first end and contact pads 112 at the second end proximate the die 92. Electrical traces 75 from the contact pads 112 to the electrical contacts 74 are formed on the board and may be covered by the solder mask or another dielectric. Electrical connections from the die 92 to the PCB 106 may be established by a wire bonding process, where small wires, which may be composed of gold or aluminum, are thermally attached to bond pads on the silicon die and to corresponding bond pads on the board. An encapsulant material 116, normally an epoxy compound, is applied to the wire bond area to protect the delicate connections from mechanical damage and other environmental effects.

The die 92 includes a plurality of electrical connection leads 110 that extend from one of the intermediate layers 109 down to the contact pads 112 on the circuit PCB 106. At least one lead couples to a single contact pad 112. Openings 150 on the left and right side of the die 92 provide access to the intermediate layers 109 to which the leads 110 are coupled. The openings 150 pass through the nozzle plate 132 and chamber layer 148 to expose contact pads 152 that are formed on the intermediate dielectric layers. There may be one opening 150 positioned on only one side of the die 92 such that all of the leads that extend from the die extend from one side while other side remains unencumbered by the leads.

The die 92 may comprise a silicon substrate, conductive layers, and polymer layers. The silicon substrate forms the supporting structure for the other layers, and contains a channel for delivering fluid composition from the bottom of the die to the upper layers. The conductive layers are deposited on the silicon substrate, forming electrical traces with high conductivity and heaters with lower conductivity. The polymer layers form passages, firing chambers, and nozzles 130 which define the drop formation geometry.

FIGS. 20-22 include more details of the die 92. The die 92 includes a substrate 107, a plurality of intermediate layers 109, and a nozzle plate 132. The nozzle plate 132 includes an outer surface 133 that subtends a surface area. The plurality of intermediate layers 109 include dielectric layers and a chamber layer 148 that are positioned between the substrate and the nozzle plate 132. The nozzle plate 132 may be about 12 microns thick.

The nozzle plate 132 may include about 4-100 nozzles 130, or about 6-80 nozzles, or about 8-64 nozzles. For illustrative purposes only, there are eighteen nozzles 130 shown through the nozzle plate 132, nine nozzles on each side of a center line. Each nozzle 130 may deliver about 0.5 to about 20 picoliters, or about 1 to about 10 picoliters, or about 2 to about 6 picoliters of a fluid composition per electrical firing pulse. The volume of fluid composition delivered from each nozzle per electrical firing pulse may be analyzed using image-based drop analysis where strobe illumination is coordinated in time with the production of drops, one example of which is the JetXpert system, available from ImageXpert, INc. of Nashua, N.H., with the droplets measured at a distance of 1-3 mm from the top of the die. The nozzles 130 may be positioned about 60 um to about 110 μm apart. Twenty nozzles 130 may be present in a 3 mm² area. The nozzles 130 may have a diameter of about 5 μm to about 40 μm, or 10 μm to about 30 μm, or about 20 μm to about 30 μm, or about 13 μm to about 25 μm. FIG. 18 is a top down isometric view of the die 92 with the nozzle plate 132 removed, such that the chamber layer 148 is exposed.

Each nozzle 130 is in fluid communication with the fluid composition in the reservoir 50 by a fluid path. Referring to FIG. 16 and FIGS. 20 and 21, the fluid path from the reservoir 50 extends from the first end 82 of the fluid transport member 80 to the second end 84 of the transport member, through the chamber 88, through the first through-hole 90, through the opening 78 of the PCB 106, through an inlet 94 of the die 92, through a channel 126, and then through the chamber 128, and out of the nozzle 130 of the die.

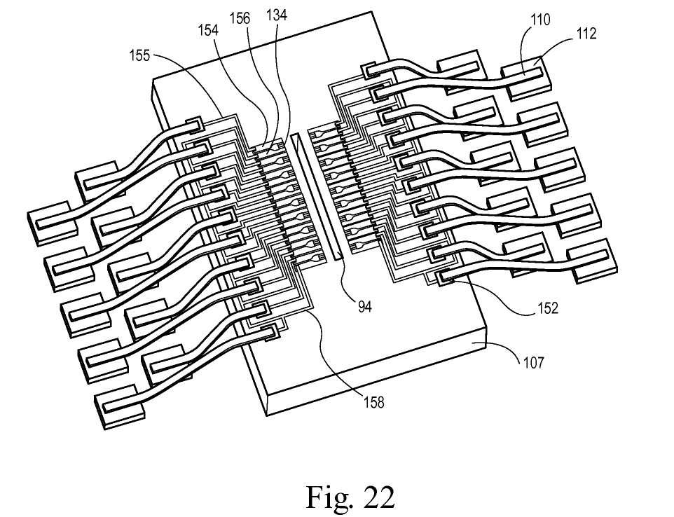

Proximate each nozzle chamber 128 is a heating element 134 (see FIG. 22) that is electrically coupled to and activated by an electrical signal being provided by one of the contact pads 152 of the die 92. Referring to FIG. 22, each heating element 134 is coupled to a first contact 154 and a second contact 156. The first contact 154 is coupled to a respective one of the contact pads 152 on the die by a conductive trace 155. The second contact 156 is coupled to a ground line 158 that is shared with each of the second contacts 156 on one side of the die. There may be only a single ground line that is shared by contacts on both sides of the die. Although FIG. 22 is illustrated as though all of the features are on a single layer, they may be formed on several stacked layers of dielectric and conductive material. Further, while the illustrated embodiment shows a heating element 134 as the activation element, the die 92 may comprise piezoelectric actuators in each chamber 128 to dispense the fluid composition from the die.

### Outer Cover

With reference back to FIGS. 6-10, the cartridge 26 includes an outer cover 40. The outer cover 40 may be defined by an interior 49 and an exterior 63. The outer cover 40 may include a top 41 that is defined by a perimeter 43. The top 41 includes an orifice 42. The top 41 of the outer cover 40 may substantially cover the top portion 51 of the reservoir 50. The orifice 42 may be disposed adjacent to the die 92.

The outer cover 40 is connected with the reservoir 50 such that a gap is formed between the outer cover 40 and the reservoir 50, forming an air flow path 46 between the outer cover 40 and the reservoir 50. In a configuration comprising a fan, the air flow path 46 allows air from the fan 32 to combine with the fluid composition 52 dispensed from the microfluidic delivery member 64 and to for the fluid composition 52 out of the orifice 42 and into the air. The restricting the air flow and the dispensed fluid composition 52 to flowing through the orifice 42 can maximize the velocity of the fluid composition 52 dispensed from the cartridge 26. The greater the velocity of the fluid composition 52 dispensed from the cartridge 26, the greater the distance the fluid composition 52 will be able to travel into the air; thus, the velocity of the fluid composition 52 can positively impact the dispersion of the fluid composition 52 into a room or space. The size of the orifice 42 can directly impact the velocity of the fluid composition 52.

The outer cover 40 may include a skirt 45 that extends from the perimeter 43 of the top 41 toward the reservoir 50. The skirt 45 may surround at least a portion of the sidewall(s) 61 of the reservoir 50. The skirt 45 may be configured such that air is able to flow longitudinally adjacent to the sidewall(s) 61 of the reservoir 50. Moreover, directing the air flow from the fan 32 longitudinally through air flow path 46 substantially 360 degrees around the sidewall 61 allows for a uniform flow of air from the skirt 45 to the orifice 42, minimizing the opportunity for turbulence to form inside of the outer cover 40 that could cause dispensed fluid composition 52 to become trapped in the air flow path 46 and possibly redeposited onto the die 92.

The skirt 45 may cover at least a portion of the microfluidic delivery member 64. The skirt 45 may cover the entire microfluidic delivery member 64. Covering the electrical contacts 74 and the die 92 of the microfluidic delivery member 64 can prevent damage that may be caused by a user touching the electrical contacts 74 and/or die 92. For example, oil and/or dirt on a user's hands can clog the die 92 and prevent fluid composition from releasing through the nozzles 130 of the die 92. Also, oil and/or dirt on a user's hands can damage the electrical contacts 74 can decrease the strength of the electrical connection between the electrical contacts 74 on the microfluidic delivery member 64 and the electrical contacts 48 on the housing 12. Moreover, the skirt 45 of the outer cover 40 provides a safe and/or ergonomic surface for a user to grasp as the user inserts and removes the cartridge 26 from the housing 12 without damaging the microfluidic delivery member 64. The outer cover 40 can also increase the aesthetic look of the cartridge 26, as an exposed microfluidic delivery member 64 may not be aesthetically pleasing for a user.

The orifice 42 may expose at least a portion of, or substantially all of, or all of, the die 92 to the exterior 63 of the outer cover 40. By exposing at least a portion of the die 92 to the exterior 63 of the outer cover 40, the fluid composition dispensed from the die 92 is unrestricted as it passes through the orifice 42. As a result, deposition of fluid composition onto the outer cover 40 as it is dispensed from the die 92 may be kept to a minimum or even prevented.

The outer cover 40 may be configured such that air flow through the air flow path 46 continually increases in pressure from the skirt 45 to the orifice 42. It is to be appreciated that if the pressure through the air flow path 46 is increased and then decreased before the air exits the orifice 42, turbulence may be formed that reduce the air flow out of the orifice 42 or cause fluid composition 52 to become trapped in the air flow path 46 or on the top portion 51 of the reservoir 50. An exemplary outer cover is described in U.S. patent application entitled “MICROFLUIDIC DELIVERY SYSTEM AND CARTRIDGE HAVING AN OUTER COVER”, Attorney Docket No. 14016, filed on Sep. 16, 2015 and U.S. patent application entitled “MICROFLUIDIC DELIVERY SYSTEM AND CARTRIDGE HAVING AN OUTER COVER”, Attorney Docket No. 14017, filed on Sep. 16, 2015.

### Sensors

The delivery system may include commercially available sensors that respond to environmental stimuli such as light, noise, motion, and/or odor levels in the air. For example, the delivery system can be programmed to turn on when it senses light, and/or to turn off when it senses no light. In another example, the delivery system can turn on when the sensor senses a person moving into the vicinity of the sensor. Sensors may also be used to monitor the odor levels in the air. The odor sensor can be used to turn-on the delivery system, increase the heat or fan speed, and/or step-up the delivery of the fluid composition from the delivery system when it is needed.

VOC sensors can be used to measure intensity of perfume from adjacent or remote devices and alter the operational conditions to work synergistically with other perfume devices. For example a remote sensor could detect distance from the emitting device as well as fragrance intensity and then provide feedback to device on where to locate device to maximize room fill and/or provide the “desired” intensity in the room for the user.

The devices may communicate with each other and coordinate operations in order to work synergistically with other perfume devices.

The sensor may also be used to measure fluid composition levels in the reservoir or count firing of the heating elements to indicate the cartridge's end-of-life in advance of depletion. In such case, an LED light may turn on to indicate the reservoir needs to be filled or replaced with a new reservoir.

The sensors may be integral with the delivery system housing or in a remote location (i.e. physically separated from the delivery system housing) such as remote computer or mobile smart device/phone. The sensors may communicate with the delivery system remotely via low energy blue tooth, 6 low pan radios or any other means of wirelessly communicating with a device and/or a controller (e.g. smart phone or computer).

The user may be able to change the operational condition of the device remotely via low energy blue tooth, or other means.

### Smart Chip

The cartridge 26 may include a memory in order to transmit optimal operational condition to the device.

### Fluid Composition

To operate satisfactorily in a microfluidic delivery system, many characteristics of a fluid composition are taken into consideration. Some factors include formulating fluid compositions with viscosities that are optimal to emit from the microfluidic delivery member, formulating fluid compositions with limited amounts or no suspended solids that would clog the microfluidic delivery member, formulating fluid compositions to be sufficiently stable to not dry and clog the microfluidic delivery member, etc. Operating satisfactorily in a microfluidic delivery system, however, addresses only some of the requirements necessary for a fluid composition having more than 50 wt. % of a perfume mixture to atomize properly from a microfluidic delivery member and to be delivered effectively as an air freshening or malodor reducing composition.

The fluid composition may exhibit a viscosity of less than 20 centipoise (“cps”), alternatively less than 18 cps, alternatively less than 16 cps, alternatively from about 5 cps to about 16 cps, alternatively about 8 cps to about 15 cps. And, the volatile composition may have surface tensions below about 35, alternatively from about 20 to about 30 dynes per centimeter. Viscosity is in cps, as determined using the Bohlin CVO Rheometer system in conjunction with a high sensitivity double gap geometry.

The fluid composition is free of suspended solids or solid particles existing in a mixture wherein particulate matter is dispersed within a liquid matrix. Free of suspended solids is distinguishable from dissolved solids that are characteristic of some perfume materials.

The fluid composition may comprise volatile materials. Exemplary volatile materials include perfume materials, volatile dyes, materials that function as insecticides, essential oils or materials that acts to condition, modify, or otherwise modify the environment (e.g. to assist with sleep, wake, respiratory health, and like conditions), deodorants or malodor control compositions (e.g. odor neutralizing materials such as reactive aldehydes (as disclosed in U.S. 2005/0124512), odor blocking materials, odor masking materials, or sensory modifying materials such as ionones (also disclosed in U.S. 2005/0124512)).

The volatile materials may be present in an amount greater than about 50%, alternatively greater than about 60%, alternatively greater than about 70%, alternatively greater than about 75%, alternatively greater than about 80%, alternatively from about 50% to about 100%, alternatively from about 60% to about 100%, alternatively from about 70% to about 100%, alternatively from about 80% to about 100%, alternatively from about 90% to about 100%, by weight of the fluid composition.

The fluid composition may contain one or more volatile materials selected by the material's boiling point (“B.P.”). The B.P. referred to herein is measured under normal standard pressure of 760 mm Hg. The B.P. of many perfume ingredients, at standard 760 mm Hg can be found in “Perfume and Flavor Chemicals (Aroma Chemicals),” written and published by Steffen Arctander, 1969.

The fluid composition may include a perfume mixture of one or more perfume materials. The perfume mixture may have an average boiling point of less than 275° C., alternatively less than 250° C., alternatively less than 220° C., alternatively less than about 180° C., alternatively about 70° C. to about 250° C. A quantity of low B.P. ingredients (<200° C.) in the perfume mixture can be used to help higher boiling point formulations to be ejected. A fluid composition with a boiling point above 250° C. could be made to eject with good performance if the fluid composition comprises from about 50% to about 100%, or about 60% to about 100%, or about 75% to about 100%, by weight of the fluid composition, of a perfume mixture of volatile perfume materials, wherein the perfume mixture has an average boiling point of less than 250° C., or less than 225° C. despite the overall average of the fluid composition still being above 250° C.

The fluid composition may comprise, consist essentially of, or consist of volatile perfume materials.

Tables 2 and 3 outline technical data on perfume materials suitable for the present fluid composition 52. Approximately 10%, by weight of the fluid composition, may be ethanol, which may be used as a diluent to reduce boiling point to a level less than 250° C. Flash point may be considered in choosing the perfume formulation as flash points less than 70° C. require special shipping and handling in some countries due to flammability. Hence, there may be advantages to formulate to higher flash points.

Table 2 lists some non-limiting, exemplary individual perfume materials suitable for the present fluid composition.

TABLE 2

| CAS Number | Perfume Raw Material Name | B.P. (° C.) |
|---|---|---|
| 105-37-3 | Ethyl propionate | 99 |
| 110-19-0 | Isobutyl acetate | 116 |
| 928-96-1 | Beta gamma hexenol | 157 |
| 80-56-8 | Alpha Pinene | 157 |
| 127-91-3 | Beta Pinene | 166 |
| 1708-82-3 | cis-hexenyl acetate | 169 |
| 124-13-0 | Octanal | 170 |
| 470-82-6 | Eucalyptol | 175 |
| 141-78-6 | Ethyl acetate | 77 |

Table 3 shows an exemplary perfume mixture having a total B.P. less than 200° C.

TABLE 3

| CAS Number | Perfume Raw Material Name | Wt % | B.P. (° C.) |
|---|---|---|---|
| 123-68-2 | Allyl Caproate | 2.50 | 185 |
| 140-11-4 | Benzyl Acetate | 3.00 | 214 |
| 928-96-1 | Beta Gamma Hexenol | 9.00 | 157 |
| 18479-58-8 | Dihydro Myrcenol | 5.00 | 198 |
| 39255-32-8 | Ethyl 2 Methyl Pentanoate | 9.00 | 157 |
| 77-83-8 | Ethyl Methyl Phenyl Glycidate | 2.00 | 260 |
| 7452-79-1 | Ethyl-2-Methyl Butyrate | 8.00 | 132 |
| 142-92-7 | Hexyl Acetate | 12.50 | 146 |
| 68514-75-0 | Orange Phase Oil 25X1.18%-Low | 10.00 | 177 |
| | Cit. 14638 | | |
| 93-58-3 | Methyl Benzoate | 0.50 | 200 |
| 104-93-8 | Para Cresyl Methyl Ether | 0.20 | 176 |
| 1191-16-8 | Prenyl Acetate | 8.00 | 145 |
| 88-41-5 | Verdox | 3.00 | 223 |
| 58430-94-7 | Iso Nonyl Acetate | 27.30 | 225 |
| | TOTAL: | 100.00 | |

The fluid composition may also include solvents, diluents, extenders, fixatives, thickeners, or the like. Non-limiting examples of these materials are ethyl alcohol, carbitol, diethylene glycol, dipropylene glycol, diethyl phthalate, triethyl citrate, isopropyl myristate, ethyl cellulose, and benzyl benzoate.

The fluid composition may contain functional perfume components (“FPCs”). FPCs are a class of perfume raw materials with evaporation properties that are similar to traditional organic solvents or volatile organic compounds (“VOCs”). “VOCs”, as used herein, means volatile organic compounds that have a vapor pressure of greater than 0.2 mm Hg measured at 20° C. and aid in perfume evaporation. Exemplary VOCs include the following organic solvents: dipropylene glycol methyl ether (“DPM”), 3-methoxy-3-methyl-1-butanol (“MMB”), volatile silicone oil, and dipropylene glycol esters of methyl, ethyl, propyl, butyl, ethylene glycol methyl ether, ethylene glycol ethyl ether, diethylene glycol methyl ether, diethylene glycol ethyl ether, or any VOC under the tradename of Dowanol™ glycol ether. VOCs are commonly used at levels greater than 20% in a fluid composition to aid in perfume evaporation.

The FPCs of the present fluid composition aid in the evaporation of perfume materials and may provide a hedonic, fragrance benefit. FPCs may be used in relatively large concentrations without negatively impacting perfume character of the overall composition. As such, The fluid composition may be substantially free of VOCs, meaning it has no more than 18%, alternatively no more than 6%, alternatively no more than 5%, alternatively no more than 1%, alternatively no more than 0.5%, by weight of the composition, of VOCs. The volatile composition may be free of VOCs.

Perfume materials that are suitable as FPCs are disclosed in U.S. Pat. No. 8,338,346.

### Method of Operation

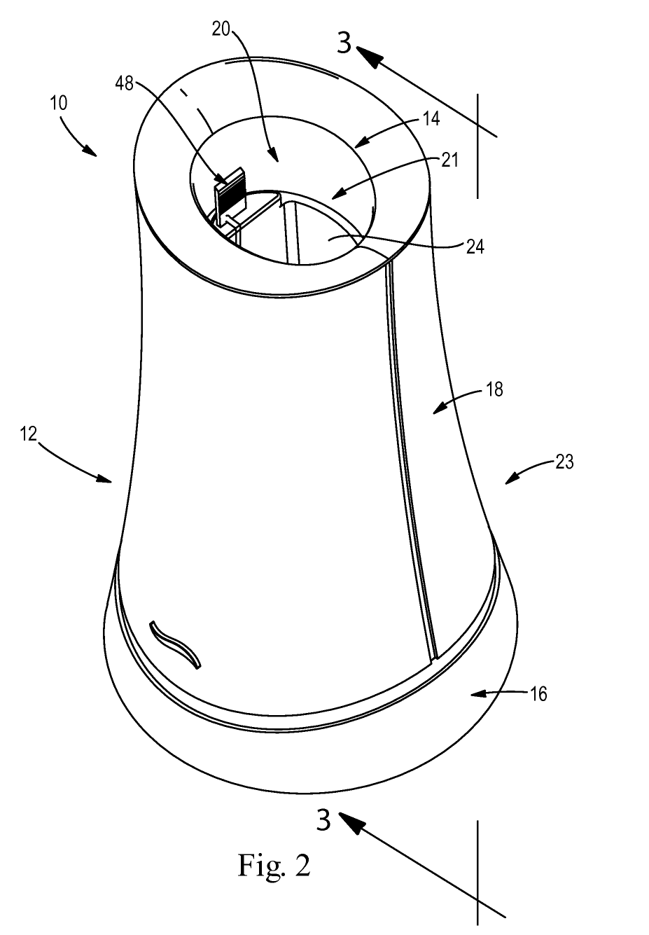

With reference to FIGS. 2-4 and 6-8, the microfluidic delivery system 10 may deliver a fluid composition 52 from the cartridge 26 using thermal heating or vibration via piezoelectric crystals, for example. The capillary tube 80 directs fluid composition 52 contained within the reservoir 50 toward the die 92 of the microfluidic delivery member 64. The capillary tube 80 may be configured to direct the fluid composition 52 up, opposite the force of gravity to the die 92. After passing through the second end portion 84 of the capillary tube 80, the fluid composition 52 travels through the die 92.

In a microfluidic delivery system that utilizes thermal inkjet technology, the fluid composition 52 travels through the fluid channel 156 and into the inlet 184 of each fluid chamber 180. The fluid composition 52, which may comprise in part a volatile component, travels through each fluid chamber 128 to the heater 134 of each fluid chamber 128. The heater 134 vaporizes at least a portion of the volatile components in the fluid composition 52, causing a vapor bubble form. The expansion created by the vapor bubble causes a droplet of fluid composition 52 to be ejected through the nozzle 130. The vapor bubble then collapses and causes the droplet of fluid composition 52 to break away and release from the orifice 130. The fluid composition 52 then refills the fluid chamber 128 and the process may be repeated to atomize additional droplets of fluid composition 52.

In a configuration comprising a fan, the fan 32 pulls air from the air inlet(s) 27 into the interior 21 of the housing in order to pressurize the air in the interior 21 of the housing 12. Because fluid will travel from an area of high pressure to an area of low pressure, the air in the interior 21 of the housing 12 will follow the least restrictive path to reach the exterior 23 of the housing 12. As a result, in a configuration comprising an outer cover 40, the housing 12 may be configured such that the pressurized air in the interior 21 of the housing 12 flows through the air flow channel 34 between the holder 24 and the upper portion 14 of the housing 12. From the air flow channel 34, the pressurized air will flow through the air flow path 46 between the outer cover 40 and the reservoir 50. If the outer cover 40 of the cartridge 26 is not sealably engaged with the housing 12, some air may escape through the gap between the outer cover 40 and the housing 12. The air flow through the gap between the outer cover 40 and the housing 12 may be reduced by configuring the flow path through the air flow channel 34 and the air flow path 46 to be the path of least resistance to the exterior 23 of the housing 12.

The air flowing through the air flow path 46 combines with the fluid composition 52 that was atomized from the microfluidic delivery member 64. Then, the combined fluid composition 52 and air flow exit out of the orifice 42 of the outer cover 40. The shape of the air flow path 46 may direct the air out of the orifice 42 in the same or substantially the same direction as the direction the fluid composition 52 is being dispensed from the die 92. The air provides additional force, in addition to the force of dispensing the atomized fluid composition 52 from the microfluidic delivery member 64, to direct the fluid composition 52 into the air or onto a target surface.

Other ejection processes may be used in addition or in the alternative to heaters used to atomize the fluid composition 52. For instance, piezoelectric crystal elements or ultrasonic fluid ejection elements may be used to atomize the fluid composition from the die 92.

The output of the microfluidic delivery system 10 may be adjustable or programmable. For example, the timing between releases of droplets of fluid composition 52 from the microfluidic delivery system 10 may be any desired timing and can be predetermined or adjustable. Further, the flow rate of fluid composition released from the microfluidic delivery system 10 can be predetermined or adjustable. For example, the microfluidic delivery system 10 may be configured to deliver a predetermined amount of the fluid composition 52, such as a perfume, based on a room size or may be configured to be adjustable as desired by the user. For exemplary purposes only, the flow rate of fluid composition 52 released from the cartridge 26 could be in the range of about 5 to about 60 mg/hour or any other suitable rate or range.

The microfluidic delivery system 10 may be used to deliver a fluid composition into the air. The microfluidic delivery system 10 may also be used to deliver a fluid composition onto a surface.

Upon depletion of the fluid composition in the reservoir 50, the microfluidic cartridge 26 may be removed from the housing 10 and replaced with another microfluidic cartridge 26.

All percentages stated herein are by weight unless otherwise specified.

Values disclosed herein as ends of ranges are not to be understood as being strictly limited to the exact numerical values recited. Instead, unless otherwise specified, each numerical range is intended to mean both the recited values, any integers within the specified range, and any ranges with the specified range. For example a range disclosed as “1 to 10” is intended to mean “1, 2, 3, 4, 5, 6, 7, 8, 9, 10.”

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A cartridge for a microfluidic delivery system, the cartridge comprising:
a reservoir for containing a fluid composition;
a capillary tube having a first end portion and a second end portion opposing the first end portion, wherein the first end portion of the capillary tube is in fluid communication with the reservoir, and wherein the second end portion of the capillary tube is operatively connected with the reservoir;
a restriction member in fluid communication with the capillary tube and configured to restrict fluid flow; and
a microfluidic delivery member connected with and in fluid communication with the reservoir, wherein the microfluidic delivery member comprises a die having at least one nozzle.

2. The cartridge of claim 1, wherein the reservoir comprises a top portion, a base portion opposing the top portion, and at least one sidewall extending between and connecting the top and base portions, wherein the capillary tube is connected with the top portion of the reservoir and extends toward the base portion of the reservoir.

3. The cartridge of claim 1, wherein the restriction member is at least partially disposed within the capillary tube.

4. The cartridge of claim 1, wherein the restriction member comprises a wick material.

5. The cartridge of claim 1, wherein a back pressure at the restriction member is in the range of about 249 Pa to about 5 kPa.

6. The cartridge of claim 1, wherein the restriction member comprise an orifice plate.

7. The cartridge of claim 1, wherein the die dispenses from about 5 mg/hour to about 60 mg/hour of fluid composition.

8. A microfluidic delivery system comprising:
a housing having a base, at least one sidewall connected with the base, and an opening for receiving a cartridge at least partially within the housing;
a cartridge releasably and electrically connectable with the housing, the cartridge comprising:
a reservoir containing a fluid composition to be dispensed from at least one nozzle;
a capillary tube having a first end portion and a second end portion opposing the first end portion, wherein the first end portion of the capillary tube is in fluid communication with the reservoir, and wherein the second end portion of the capillary tube is operatively connected with the reservoir;
a restriction member in fluid communication within the capillary tube and configured to restrict fluid flow.

9. The microfluidic delivery system of claim 8, wherein the cartridge further comprises a microfluidic delivery member connected with and in fluid communication with the reservoir, wherein the microfluidic delivery member comprises a microfluidic die having the nozzle.

10. The microfluidic delivery system of claim 8, wherein the reservoir comprises a top portion, a base portion opposing the top portion, and at least one sidewall extending between and connecting the top and base portions, wherein the capillary tube is connected with the top portion of the reservoir and extends toward the base portion of the reservoir.

11. The microfluidic delivery system of claim 8, wherein the restriction member is at least partially disposed within the capillary tube.

12. The microfluidic delivery system of claim 8, wherein the restriction member comprises a wick material.

13. The microfluidic delivery system of claim 8, wherein a back pressure at the restriction member is in the range of about 249 Pa to about 5 kPa.

14. The microfluidic delivery system of claim 8, wherein the restriction member comprise an orifice plate.

15. The microfluidic delivery system of claim 8, wherein the die dispenses from about 5 mg/hour to about 60 mg/hour of fluid composition.

16. A method of delivering a composition into the air, the method comprising the steps of:
providing a microfluidic delivery device, the microfluidic delivery device comprising a reservoir, a capillary tube in fluid communication with the reservoir, and a microfluidic delivery member in fluid communication with the capillary tube and connected with the reservoir, wherein the reservoir contains a fluid composition;
directing the fluid composition from the reservoir, through the capillary tube, and to the microfluidic deliver member;
restricting the flow of the fluid composition to a first flow rate as the fluid composition flows through a portion of the capillary tube; and
atomizing the fluid composition into the air at a second flow rate, wherein second flow rate is at least about 1.5 times greater than the first flow rate.

17. The method of claim 16, wherein the reservoir comprises a top portion, a base portion opposing the top portion, and at least one sidewall extending between and connecting the top and base portions, wherein the capillary tube is connected with the top portion of the reservoir and extends toward the base portion of the reservoir.

18. The method of claim 16, wherein the restriction member is a separate element from the capillary tube.

19. The method of claim 16, wherein the restriction member comprises a wick material.

20. The method of claim 19, wherein a back pressure at the restriction member is in the range of about 249 Pa to about 5 kPa.

21-35. (canceled)
